(19) United States
(12) Patent Application Publication
(10) Pub. No.: US 2025/0268797 A1
(43) Pub. Date: Aug. 28, 2025

(54) SYSTEM AND METHOD FOR PRECISION ORAL MEDICATION DELIVERY BASED ON REAL-TIME HEALTH MONITORING

(71) Applicant: YESHBIO SOLUTIONS PRIVATE LIMITED, HYDERABAD (IN)

(72) Inventor: YESHPAL MATHANGI, HYDERABAD (IN)

(21) Appl. No.: 19/064,837

(22) Filed: Feb. 27, 2025

(30) Foreign Application Priority Data
Feb. 27, 2024 (IN) 202441014199

(51) Int. Cl. A61J 7/00; G16H 20/13

(52) U.S. Cl. CPC A61J 7/0053; G16H 20/13; A61J 2200/70

(57) ABSTRACT

The present invention discloses a system and method for precision oral medication delivery based on real-time health monitoring. The system comprises a palatal medication delivery device, a plurality of sensors configured to measure real-time health parameters of a user, a precision medication control unit comprising a plurality of subsystems comprising a data processing subsystem configured to receive the measured real-time health parameters, filter out noise from the received real-time health parameters, normalize the noise-free health parameters, extract health features from the normalized noise-free data, an artificial intelligence (AI) subsystem configured to determine health patterns based on the extracted health features and compare the determined health patterns with a predefined baseline to detect a deviation, a dosage prediction subsystem configured to determine a precision dosage instruction, upon detection of the deviation and the digital controller unit configured to release a microfluidic dosage of medication based on the precision dosage instruction.

### EARLIEST PRIORITY DATE

This application claims priority from a Provisional patent application filed in India having patent application No. 202441014199, filed on 27 Feb. 2024 and titled “SYSTEM AND METHOD FOR DELIVERING ORAL TRANS MUCOSAL MEDICATION AND SUPPLEMENTS WITH REALTIME MONITORING”.

### FIELD OF INVENTION

Embodiments of the present invention in general relate to the technical field of medication delivery system, and more particularly, to a system and method for precision oral medication delivery based on real-time health monitoring.

### BACKGROUND

Medication delivery systems have evolved significantly, with oral administration remaining one of the most common and preferred methods due to its non-invasive nature, patient comfort, and ease of use. However, traditional oral medication delivery often struggles with achieving precise dosing, maintaining consistent drug levels, and adapting to individual patient needs. Real-time health monitoring has emerged as a promising approach to enhance medical care by providing continuous insights into a patient's health status but integrating it with medication delivery systems presents challenges in data processing, analysis, and responsive drug administration. Advancements in microfluidics, miniaturized sensors, artificial intelligence, and wireless communication technologies have addressed some of these issues.

However, integrating these advanced technologies into a precision oral medication delivery system while ensuring patient comfort, long-term reliability, and seamless operation remains a significant challenge. Additionally, the complexity of processing real-time health data, ensuring the accuracy of AI-driven dosing decisions, and maintaining the security and privacy of transmitted data are ongoing concerns. These challenges highlight the need for innovative solutions that can provide personalized, adaptive treatment based on an individual's real-time health status.

Hence, there is a need for an efficient system and method for precision oral medication delivery based on real-time health monitoring, to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with one embodiment of the present invention disclosure, a system for precision oral medication delivery based on real-time health monitoring is disclosed. The system comprising a palatal medication delivery device comprising a biocompatible layer, a medication holding chamber comprising a plurality of micro-holes that are distributed across surface of the medication holding chamber and a digital controller unit connected with the medication holding chamber configured to control release of medication through the plurality of micro-holes, a plurality of sensors operably coupled to the palatal medication delivery device configured to measure real-time health parameters of a user, wherein the real-time health parameters comprise at least one of a body temperature parameter, a heart rate parameter, an oxygen saturation level parameter, and a salivary biomarker parameter, a precision medication control unit operably coupled to the palatal medication delivery device, comprising one or more hardware processors and a memory coupled to the one or more hardware processors, wherein the memory comprises a plurality of subsystems in the form of programmable instructions executable by the one or more hardware processors, and wherein the plurality of subsystems comprises a data processing subsystem configured to receive the measured real-time health parameters, filter out, by one or more filtering techniques, noise from the received real-time health parameters, normalize, by one or more normalization techniques, the noise-free health parameters, extract, by one or more feature extraction techniques, health features from the normalized noise-free data, an artificial intelligence (AI) subsystem configured to determine, by one or more neural networks, health patterns based on the extracted health features, wherein the determined health patterns comprise at least one of a heart rate variability pattern, a glucose level fluctuation pattern, a physical activity pattern, a respiratory pattern, and a temperature level pattern, wherein the determined health patterns indicate the real-time health status of the user and compare the determined health patterns with a predefined baseline to detect a deviation in the real-time health status of the user, a dosage prediction subsystem configured to determine a precision dosage instruction, upon detection of the deviation in the real-time health status of the user and the digital controller unit configured to release a microfluidic dosage of medication based on the precision dosage instruction.

In an embodiment, the biocompatible layer is made of at least one of a medical-grade silicone, biocompatible polymers, and hydrogel.

In another embodiment, the digital controller unit is configured to adjust a rate of medication released through the micro-holes, based on the precision dosage instruction.

In another embodiment, the plurality of sensors comprises at least one of a temperature sensor for measuring body temperature, a photoplethysmography (PPG) sensor for measuring heart rate and oxygen saturation levels, an electrochemical sensor for detecting salivary biomarkers including glucose level, cortisol level, and electrolyte level and an inertial measurement unit (IMU) sensor for tracking physical activity and movement patterns.

In yet another embodiment, the dosage prediction subsystem is configured to determine the precision dosage instruction, upon detection of the deviation in the real-time health status of the user, further comprises receiving a plurality of historical patient data from a patient database, wherein the historical patient data includes at least one of a historical health parameter of the user, a historical medication dosage data, and a treatment response to the medication, determine, by one or more learning models, a medication dosage instruction based on the real-time health status of the user and the plurality of historical patient data, optimize the medication dosage instruction to determine a precision dosage instruction for the user, based on a plurality of user-specific parameters, wherein the user-specific parameters include at least one of an age, a weight, and a metabolism, and wherein the precision dosage instruction comprises one of a precise amount of medication to be released, a rate of release of the medication, and a timing of release of the medication.

In yet another embodiment, the system further comprises a user interface operably connected to the precision medication control unit configured to display real-time health status and medication dosage information to the user.

In yet another embodiment, a wireless communication subsystem configured to transmit real-time health data and dosage information to a healthcare provider.

In yet another embodiment, the one or more filtering techniques comprise at least one of a Kalman filtering technique and a Butterworth filtering technique.

In yet another embodiment, the one or more normalization techniques comprise at least one of a Min-Max Normalization and Z-Score Normalization.

In another embodiment, the wherein the one or more feature extraction techniques comprise at least one of a Fast Fourier Transform (FFT) and a wavelet analysis.

In another embodiment, the one or more neural networks comprise at least one of a convolutional neural network (CNN) and a recurrent neural network (RNN).

In yet another embodiment, the one or more learning models comprise at least one of a supervised learning model and an reinforcement learning model, wherein the supervised learning model includes at least one of a Random Forests model and a Support Vector Machines model.

In another aspect, a method for precision oral medication delivery based on real-time health monitoring is disclosed. The method comprising measuring, by a plurality of sensors, real-time health parameters of a user operably coupled to a palatal medication delivery device, wherein the real-time health parameters comprise at least one of a body temperature parameter, a heart rate parameter, an oxygen saturation level parameter, and a salivary biomarker parameter, receiving, by a data processing subsystem, the measured real-time health parameters, filtering out, by one or more filtering techniques, noise from the received real-time health parameters, normalizing, by one or more normalization techniques, the noise-free health parameters, extracting, by one or more feature extraction techniques, health features from the normalized noise-free data, determining, by one or more neural network, health patterns based on the extracted health features, wherein the determined health patterns comprise at least one of a heart rate variability pattern, a glucose level fluctuation pattern, an activity pattern, a respiratory pattern, and a temperature level pattern, and wherein the determined health patterns indicate the real-time health status of the user, comparing, by an artificial intelligence (AI) subsystem, the determined health patterns with a predefined baseline to detect a deviation in the real-time health status of the user, determining, by a dosage prediction subsystem, a precision dosage instruction upon detection of the deviation in the real-time health status of the user, releasing, by the digital controller unit, a microfluidic dosage of medication based on the precision dosage instruction.

In an embodiment, the palatal medication delivery device comprises a biocompatible layer made of at least one of a medical-grade silicone, biocompatible polymers, and hydrogel.

In yet another embodiment, the method further comprising adjusting, by the digital controller unit, a rate of medication released through the micro-holes based on the precision dosage instruction.

In yet another embodiment, the plurality of sensors comprises at least one of a temperature sensor for measuring body temperature, a photoplethysmography (PPG) sensor for measuring heart rate and oxygen saturation levels, an electrochemical sensor for detecting salivary biomarkers including glucose level, cortisol level, and electrolyte level and an inertial measurement unit (IMU) sensor for tracking physical activity and movement patterns.

In yet another embodiment, determining the precision dosage instruction, upon detection of the deviation in the real-time health status of the user, further comprises receiving a plurality of historical patient data from a patient database, wherein the historical patient data includes at least one of a historical health parameter of the user, a historical medication dosage data, and a treatment response to the medication, determining, by one or more learning model, a medication dosage instruction based on the real-time health status and the historical patient data and optimizing the medication dosage instruction to determine a precision dosage instruction for the user, based on a plurality of user-specific parameters, wherein the user-specific parameters include at least one of an age, a weight, and a metabolism, and wherein the precision dosage instruction comprises at least one of a precise amount of medication to be released, a rate of release of the medication, and a timing of release of the medication.

In yet another embodiment, the method further comprising displaying, via a user interface operably connected to a precision medication control unit, real-time health parameters and dosage information to the user.

In yet another embodiment, the method further comprising transmitting, via a wireless communication unit, real-time health data and dosage information to a healthcare provider.

In yet another embodiment, the one or more filtering techniques comprise at least one of a Kalman filtering technique and a Butterworth filtering technique.

In another embodiment, the one or more normalization techniques comprise at least one of a Min-Max Normalization and Z-Score Normalization.

In yet another embodiment, the one or more feature extraction techniques comprise at least one of a Fast Fourier Transform (FFT) and a wavelet analysis.

In yet another embodiment, the one or more neural networks comprise at least one of a convolutional neural network (CNN) and a recurrent neural network (RNN).

In yet another embodiment, the one or more learning models comprise at least one of a supervised learning model and a reinforcement learning model, wherein the supervised learning model includes at least one of a Random Forests model and a Support Vector Machines model.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 illustrates a block diagram of an exemplary operational architecture of a system for precision oral medication delivery based on real-time health monitoring, in accordance with an embodiment of the present invention.

FIG. 2 illustrates an exemplary diagram of a palatal medication delivery device, in accordance with an embodiment of the present invention.

FIG. 3 illustrates an exemplary computational environment of a system for precision oral medication delivery based on real-time health monitoring, in accordance with an embodiment of the present invention.

FIG. 4 illustrates an exemplary block diagram of the precision medication control unit, in accordance with an embodiment of the present invention.

FIG. 5 illustrates an exemplary flowchart of a method for precision oral medication delivery based on real-time health monitoring, in accordance with an embodiment of the present invention.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE PRESENT INVENTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

Embodiments of the present invention relate to a method and system for detecting intrusion in industrial control systems.

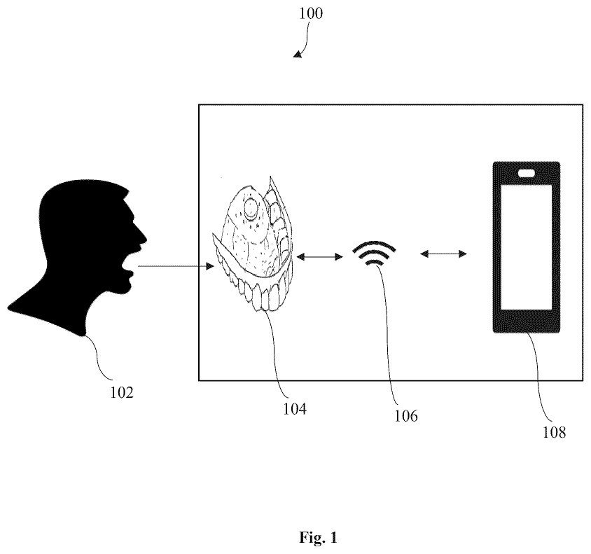

Referring now to the drawings, and more particularly to FIG. 1 through FIG. 5, where similar reference characters denote corresponding features consistently throughout the figures, there are shown preferred embodiments, and these embodiments are described in the context of the following exemplary system and/or method.

The present invention introduces a system for precision oral medication delivery based on real-time health monitoring, which is crucial for enhancing personalized treatment efficacy across various medical conditions. Leveraging advancements in microfluidics, miniaturized sensors, artificial intelligence, and wireless communication technologies, the invention integrates real-time health data with adaptive medication dosing. The system continuously monitors vital signs and physiological parameters, providing unparalleled insights into a patient's health status. By analyzing real-time data through advanced AI models, the system dynamically adjusts medication delivery to maintain optimal drug levels and adapts to individual patient needs. This precision-driven approach ensures precision dosing, improves treatment outcomes, and minimizes side effects. Its application in managing chronic conditions, such as diabetes, cardiovascular diseases, and neurological disorders, underscores its utility in delivering personalized, adaptive treatment, thereby enhancing patient comfort, safety, and overall well-being.

FIG. 1 illustrates a block diagram of an exemplary operational architecture of a system for precision oral medication delivery based on real-time health monitoring, in accordance with an embodiment of the present invention.

According to an embodiment of the present invention, a block diagram of a medication delivery system 100 is disclosed. The medication delivery system 100 may include a user 102, a palatal medication delivery device 104, a wireless communication interface 106, and a user device 108. The palatal medication delivery device 104 may be configured to deliver medication to a user.

In an embodiment, the palatal medication delivery device 104 may be used for unconscious or coma patients as a patient health stabilizer in medical-ambulatory emergencies. The palatal medication delivery device 104 may also be adapted for use as an invasive dermal or intravenous digital controlled precision drug delivery system for patients admitted in hospitals.

The wireless communication interface 106 may connect the palatal medication delivery device 104 with the user device 108. The wireless communication interface 106 may enable bidirectional data transmission between these components. The user device 108 may receive and display information from the palatal medication delivery device 104 through the wireless communication interface 106. The user device 108 may include a user interface to display health status and dosage information to the user.

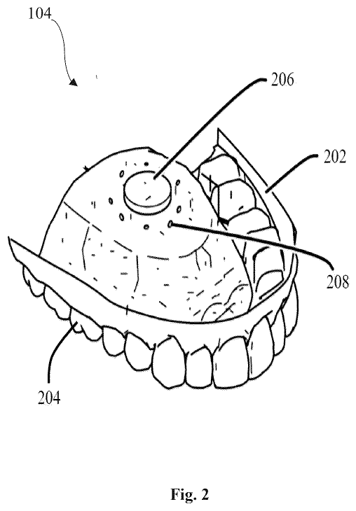

FIG. 2 illustrates an exemplary diagram of a palatal medication delivery device, in accordance with an embodiment of the present invention.

According to an embodiment of the present invention, the palatal medication delivery device 104 comprises a biocompatible layer 202 that forms the base structure of the device. In an embodiment, the biocompatible layer 202 may be made of medical-grade silicone, biocompatible polymers, or hydrogel. The design and manufacturing of the palatal medication delivery device 104 may involve several steps to ensure a customized fit for each user.

First, a 3D oral scanning process may be used to capture the precise dimensions and contours of the user's palate. This scan data may then be used in computer-aided design (CAD) software to create a virtual model of the palatal medication delivery device 104. Based on this digital design, a structure of the palatal medication delivery device 104 may be created using 3D printing with biocompatible resins. The biocompatible layer 202 incorporates teeth impressions 204 that allow the palatal medication delivery device 104 to be positioned and secured on the upper jaw. The palatal medication delivery device 104 includes a medication holding chamber 206 integrated within the structure.

In an embodiment, the medication holding chamber 206 may be divided into 3-5 separate compartments to store different medications simultaneously. The medication holding chamber 206 features micro-holes 208 distributed across its surface. These micro-holes 208 are configured to enable controlled release of medication from the medication holding chamber 206. A digital controller unit may be connected with the medication holding chamber 206 to control the release of medication through the micro-holes 208. The digital controller unit may use flexible printed circuit boards (PCBs) that conform to the palatal shape, allowing for seamless integration within the palatal medication delivery device 104.

In another embodiment, the palatal medication delivery device 104 may incorporate passive or active temperature regulation to maintain drug stability within the medication holding chamber 206. This temperature regulation may work in conjunction with the digital controller unit to ensure optimal medication storage conditions. The palatal medication delivery device 104 may include a self-sealing port for easy refilling of medication in the medication holding chamber 206. This allows for convenient replenishment of medication without the need to remove the device from the user's mouth. The palatal medication delivery device 104 may incorporate a custom-designed, flexible, thin-film lithium-ion battery as a power source.

In yet another embodiment, the palatal medication delivery device 104 may use wireless inductive charging with Qi standard or proprietary near-field magnetic resonance technology for recharging the battery. The palatal medication delivery device 104 may also include a backup capacitor for short-term critical functions during battery failure. The combination of these components and features in the palatal medication delivery device 104 allows for precise, controlled, and personalized delivery of medication directly into the oral cavity.

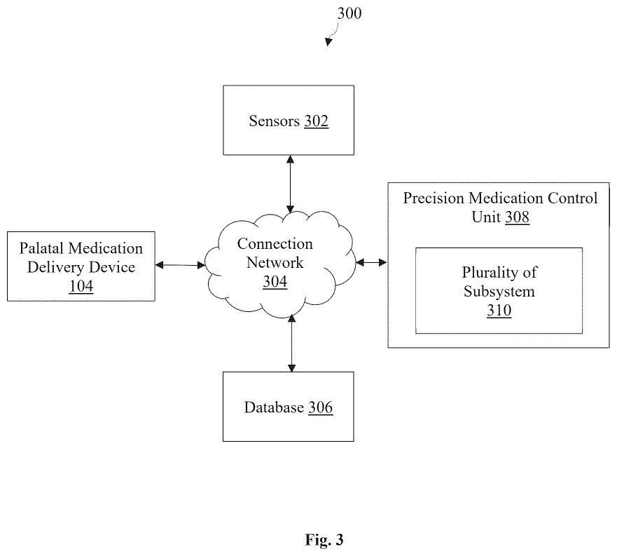

FIG. 3 illustrates an exemplary computational environment of a system for precision oral medication delivery based on real-time health monitoring, in accordance with an embodiment of the present invention.

According to an embodiment of the present invention, the medication delivery system may include a plurality of sensors 302, a connection network 304, a palatal medication delivery device 104, a database 306 and a precision medication control unit 308. The precision medication control unit 308 may contain a plurality of subsystem 310. The plurality of sensors 302 may measure the real-time health parameters of a user. The plurality of sensors 302 may include a temperature sensor for measuring body temperature, a photoplethysmography (PPG) sensor for measuring heart rate and oxygen saturation levels, an electrochemical sensor for detecting salivary biomarkers, and an inertial measurement unit (IMU) sensor for tracking physical activity and movement patterns.

The connection network 304 may facilitate communication between the components of the medication delivery system. The connection network 304 may enable data exchange between the plurality of sensors 302, the palatal medication delivery device 104, and the precision medication control unit 308. The connection network 304 may include a wired communication network, a wide area network (WAN), a metropolitan area network (MAN), a telephone network such as the public switched telephone network (PSTN) and a cellular network, an intranet, an internet, a fiber optic network, a satellite network, a cloud computing network, and a combination of networks. The connection network 304 may also include an ethernet using at least one of a transmission control protocol/internet protocol (TCP/IP), a user datagram protocol (UDP), and the like, thereby establishing a connection with a database 306. The database 306 may be configured to access and update stored information related to patient health status and medication dosage data.

The palatal medication delivery device 104 may be configured to deliver medication to the user. The palatal medication delivery device 104 may include a digital controller unit. The digital controller unit may be encapsulated within a biocompatible layer using over-molding techniques. The digital controller unit may use application-specific integrated circuits (ASICs) and system-on-chip (SoC) technology for miniaturization. The digital controller unit may adjust the rate of medication release through micro-holes in the palatal medication delivery device 104. The precision medication control unit 308 may include a plurality of subsystems 310. The plurality of subsystem 310 within the precision medication control unit 308 may process information received through the connection network 304.

In an embodiment, the precision medication control unit 308 may determine dosage instructions based on the processed information. The medication delivery system may be connected to connection network 304 to enable communication with the user device 108. The user device 108 includes a variety of devices such as laptop computers, desktop computers, tablet computers, smartphones, wearable devices, and digital cameras. The precision medication control unit 308 may personalize the medication delivery based on user-specific information.

The system 300 employs a star-like topology, with the connection network 304 serving as the central connection point between the palatal medication delivery device 104, the precision medication control unit 308, the plurality of sensors 302, and the database 306. This topology ensures efficient and reliable communication, enabling real-time monitoring of the health status of the user and precision oral medication delivery.

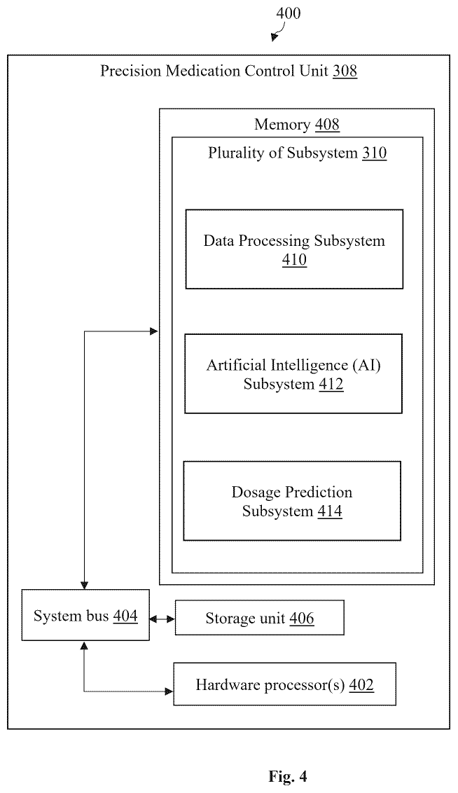

FIG. 4 illustrates an exemplary block diagram 400 of the precision medication control unit, in accordance with an embodiment of the present invention.

In accordance with an embodiment of the present invention, the precision medication control unit 308 may include a memory 408, a system bus 404, a storage unit 406, and one or more hardware processors 402. The memory 408 may contain a plurality of subsystems 310, including a data processing subsystem 410, an artificial intelligence (AI) subsystem 412, and a dosage prediction subsystem 414.

The memory 408 may be non-transitory volatile memory and non-volatile memory. The memory 408 may be coupled for communication with the one or more hardware processors 402, such as being a computer-readable storage medium. The one or more hardware processors 402 may execute machine-readable instructions and/or source code stored in the memory 408. A variety of machine-readable instructions may be stored in and accessed from the memory 408. The memory 408 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory 408 includes the plurality of subsystems 310 stored in the form of machine-readable instructions on any of the above-mentioned storage media and may be in communication with and executed by the one or more hardware processors 402.

In an embodiment, the one or more hardware processors 402, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 402 may also include embedded controllers, such as generic or programmable logic devices or arrays, application-specific integrated circuits, single-chip computers, and the like.

The system bus 404 may facilitate communication between the memory 408, the storage unit 406, and the one or more hardware processor(s) 402. The one or more hardware processor(s) 402 may execute instructions stored in memory 408 to coordinate the operations of the plurality of subsystems 310.

In accordance with the embodiment, the plurality of sensors 302 measure the real-time health parameters of a user. The plurality of sensors 302 include a temperature sensor for measuring body temperature, a photoplethysmography (PPG) sensor for measuring heart rate and oxygen saturation levels, an electrochemical sensor for detecting salivary biomarkers, and an inertial measurement unit (IMU) sensor for tracking physical activity and movement patterns.

In accordance with the embodiment, the plurality of subsystem 310 comprises a data processing subsystem 410 configured to receive the measured real-time health parameters of the user. The data processing subsystem 410 then employs filtering methods like Kalman filtering and Butterworth filtering for removing noise from the received health parameters. The Kalman filtering technique is a recursive technique that estimates unknown variables from noisy measurements by maintaining a probabilistic estimate of the system state, which is updated with each new measurement. For the received real-time health parameters, Kalman filtering smooths out fluctuations in parameters like heart rate or oxygen saturation levels, providing a more stable and accurate representation of the user's physiological state.

In another embodiment, the data processing subsystem 410 may also employ the Butterworth filtering technique, known for its maximally flat frequency response in the passband and is ideal for separating desired signal components from unwanted noise without distorting the signal's amplitude. This technique is used to remove high-frequency noise from sensor readings or to isolate specific frequency bands of interest in signals like heart rate variability or respiratory waveforms.

In an embodiment, the data processing subsystem 410 may also combine these two filtering techniques to effectively remove noise and artifacts from the received health parameters, ensuring that subsequent analyses and decision-making processes are based on clean, reliable data.

The data processing subsystem 410 after, filtering the noise from the real-time health parameters, utilizes normalization techniques to standardize the diverse health parameters. Two normalization techniques may be employed: Min-Max Normalization and Z-Score Normalization. The Min-Max Normalization technique scales all values to a fixed range, typically between 0 and 1, by subtracting the minimum value from each data point and dividing by the range (maximum-minimum) of the dataset. This ensures that all health parameters, regardless of their original units or ranges, can be directly compared and analyzed together. For example, body temperature and heart rate can be normalized to the same scale for uniform analysis.

In another embodiment, the data processing subsystem 410 may employ the Z-Score Normalization technique. The technique transforms the noise-free health parameters to have a mean of 0 and a standard deviation of 1 by subtracting the mean from each data point and dividing by the standard deviation. This helps mitigate the impact of outliers and ensures that extreme values do not disproportionately influence the analysis or decision-making processes.

In accordance with the embodiment, the data processing subsystem 410 further employs advanced feature extraction techniques to extract health features from the normalized health parameters. The data processing subsystem 410 utilizes feature extraction techniques, such as the Fast Fourier Transform (FFT), to convert signals into their corresponding frequency-domain representations. This enables the data processing subsystem 410 to identify dominant frequencies, harmonic structures, and spectral characteristics indicative of specific health conditions or physiological states. Specifically, the FFT is applied to the normalized health data, including measurements such as body temperature, heart rate, oxygen saturation levels, and salivary biomarkers. For example, FFT can be used to extract features from heart rate variability data, which reveal the balance between sympathetic and parasympathetic nervous system activity, providing insights into stress levels and cardiovascular health. This technique is applied to various physiological signals such as ECGs, EEGs, or respiratory waveforms, enabling the extraction of features that provide insights into stress levels, cardiovascular health, and overall autonomic function.

In another embodiment, the data processing subsystem 410 may also utilize the wavelet analysis technique for feature extraction from the normalized health parameter. This technique provides both frequency and temporal information about the signals by decomposing the normalized health data into a series of wavelets at different scales and positions. Wavelet analysis is particularly effective for analyzing non-stationary signals common in biological systems, such as those collected from sensors measuring body temperature, heart rate, oxygen saturation levels, and salivary biomarkers. By capturing both high-frequency details and low-frequency trends simultaneously, wavelet analysis enables the extraction of features that detect transient events or abrupt changes in physiological signals. For example, it can extract features related to rapid spikes or drops in heart rate, sudden fluctuations in oxygen saturation levels, or abrupt changes in salivary biomarkers like glucose or cortisol levels.

The specific feature being extracted by the data processing subsystem 410 depends on the category of health parameter being processed. For instance, in the case of heart rate parameters, dominant frequencies are identified using Fast Fourier Transform (FFT) to detect the primary heart rate rhythm and its harmonics, while heart rate variability (HRV) is extracted to assess the balance between the sympathetic and parasympathetic nervous systems. Transient events, such as arrhythmias or sudden spikes/drops in heart rate, are detected using wavelet analysis. Similarly, for oxygen saturation level parameters, spectral characteristics indicating respiratory health or issues like hypoxemia are extracted using FFT.

Body temperature parameters are analyzed to extract features related to cyclical patterns, such as circadian rhythms or fever patterns, using FFT, while features related to rapid changes in temperature, which could indicate infection, inflammation, or other acute conditions, are extracted using wavelet analysis. In the case of salivary biomarkers like glucose, cortisol, and electrolytes, features related to glucose level fluctuations are extracted using FFT, while features related to cortisol level trends are extracted using wavelet analysis. Features related to electrolyte imbalances are identified using spectral analysis.

Respiratory waveforms are processed to extract features related to breathing patterns using FFT, identifying dominant frequencies that may indicate normal or abnormal respiratory rhythms. Features related to apnea or hypopnea events, which are critical for diagnosing conditions like sleep apnea, are extracted using wavelet analysis. For electrocardiogram (ECG) parameter, features related to the QRS complex, including the duration and amplitude of the QRS complex, are extracted to analyze the electrical activity of the heart. Features related to the ST segment and T-wave are extracted to detect ischemia, arrhythmias, or other cardiac abnormalities, and heart rate variability (HRV) is extracted to assess autonomic function and stress levels.

Electroencephalogram (EEG) parameter is analyzed to extract features related to brainwave frequencies using FFT, including alpha, beta, theta, and delta waves, which are associated with different cognitive states and neurological conditions. Features related to event-related potentials (ERPs) are extracted to study brain responses to specific stimuli, useful in diagnosing neurological disorders, and features related to seizure activity are extracted using wavelet analysis. Physical activity data, such as that collected from an inertial measurement unit (IMU), is processed to extract features related to movement patterns using FFT, identifying repetitive movements like walking or running. Features related to activity intensity are extracted using wavelet analysis, and features related to fall detection are extracted to identify sudden, uncontrolled movements indicative of falls or accidents.

Photoplethysmography (PPG) parameter is analyzed to extract features related to pulse waveform characteristics, providing insights into cardiovascular health by examining the shape and timing of the pulse wave. Features related to pulse rate variability (PRV) are extracted to assess autonomic function and stress levels, similar to HRV, and features related to oxygen saturation trends are extracted to monitor changes in blood oxygen levels, which are critical for respiratory and cardiovascular health. By extracting these specific features, the parameter processing subsystem transforms raw sensor parameters into a rich set of extracted features (quantitative metrics) that provide a comprehensive view of the user's real-time health status.

In accordance with the embodiment, the plurality of subsystems 310 further comprises an artificial intelligence (AI) subsystem 412. The artificial intelligence (AI) subsystem 412 is configured to determine health patterns from the extracted health features. The artificial intelligence (AI) subsystem 412 processes the extracted health features to identify intricate health patterns. The artificial intelligence subsystem 412 utilizes specific neural networks such as convolutional neural networks (CNNs) and recurrent neural networks (RNNs), each serving distinct purposes in the analysis of extracted health features.

Convolutional neural networks (CNNs) are specialized architectures designed to recognize spatial patterns within extracted features. CNNs are applied to analyze complex physiological signals such as electrocardiograms (ECGs), photoplethysmography (PPG) waveforms, respiratory waveforms, and even salivary biomarker fluctuations. For example, CNNs analyze ECG waveforms by applying a series of convolutional filters that detect patterns such as the QRS complex, T-waves, and P-waves. These filters slide over the input data, extracting spatial hierarchies of features at multiple levels of abstraction. By stacking multiple convolutional layers, the CNN can learn increasingly complex representations of the signal, allowing it to identify irregularities in the shape or timing of the waveforms, which may indicate conditions like arrhythmias, ischemia, or other cardiac abnormalities.

In accordance with the embodiment, CNNs also analyze PPG waveforms to detect subtle changes in pulse morphology, such as variations in the systolic or diastolic peaks, which may indicate cardiovascular issues like hypertension or arterial stiffness. In the case of respiratory waveforms, CNNs identify patterns in breathing rhythms, such as abnormalities in the inhalation-exhalation cycle, which could signal conditions like sleep apnea or chronic obstructive pulmonary disease (COPD). For salivary biomarkers, CNNs analyze spectral representations of glucose, cortisol, or electrolyte levels to detect spatial patterns that may indicate metabolic imbalances or stress-related changes.

In another embodiment, the artificial intelligence (AI) subsystem 412 utilizes recurrent neural networks (RNNs). These RNNs are particularly adept at processing sequential data and capturing temporal dependencies. This makes them ideal for analyzing time-series health data, such as continuous glucose monitoring readings, heart rate variability, long-term temperature fluctuations, or even physical activity patterns. RNNs process sequential data by maintaining a hidden state that evolves over time, allowing the network to capture dependencies between data points that are temporally separated. For instance, when analyzing heart rate variability data, RNNs learn to recognize patterns that occur over extended periods, such as gradual increases or decreases in heart rate that may precede a hypoglycemic event or indicate stress. The network's ability to retain information from previous time steps enables it to detect long-term trends or cyclical patterns, making it well-suited for monitoring chronic conditions or tracking slow-onset health changes.

In the case of continuous glucose monitoring, RNNs analyze the sequential data to identify patterns such as postprandial spikes, nocturnal hypoglycemia, or long-term trends that may indicate insulin resistance or other metabolic issues. For temperature data, RNNs detect circadian rhythms or deviations from normal temperature ranges, which could signal infections, inflammation, or other acute conditions. When analyzing physical activity data, RNNs identify patterns in movement intensity, duration, or frequency, which may correlate with energy expenditure, physical fitness, or even fall risk.

For salivary biomarkers like glucose and cortisol, RNNs analyze the temporal dynamics of these parameters to detect patterns that may indicate metabolic stress, infection, or hormonal imbalances. For instance, RNNs can identify gradual increases in cortisol levels that may signal chronic stress or abrupt changes that could indicate an acute stress response. Similarly, RNNs analyze electrolyte fluctuations over time to detect patterns that may indicate dehydration, kidney issues, or other metabolic imbalances.

In an exemplary embodiment, the artificial intelligence (AI) subsystem 412 also employs advanced RNN architectures, such as Long Short-Term Memory (LSTM) networks and Gated Recurrent Units (GRUs) to better capture long-term dependencies in sequential data. These architectures effectively analyze complex time-series data, such as salivary biomarker fluctuations or respiratory patterns, where understanding the temporal context is critical for accurate pattern recognition.

The LSTM networks learn to recognize patterns in glucose levels that occur over days or weeks, such as gradual increases in postprandial glucose levels that may indicate insulin resistance or abrupt changes that could signal an acute metabolic issue. Similarly, GRUs effectively analyze time-series data with shorter-term dependencies, such as heart rate variability or physical activity patterns. For example, a GRU network can learn to recognize patterns in heart rate variability that occur over hours or days, such as gradual increases in heart rate that may indicate stress or abrupt changes that could signal a cardiac event.

In the case of salivary biomarkers, LSTMs and GRUs analyze the temporal dynamics of glucose, cortisol, and electrolyte levels to detect patterns that may indicate metabolic imbalances, stress, or hormonal issues. For instance, an LSTM network can identify long-term trends in cortisol levels that may signal chronic stress, while a GRU network can detect short-term fluctuations in electrolyte levels that may indicate dehydration or kidney issues. Similarly, these networks can analyze respiratory waveforms to identify patterns in breathing rhythms, such as apnea or hypopnea events, which could signal conditions like sleep apnea or COPD.

The combination of CNNs and RNNs allows the AI subsystem to perform both spatial and temporal analysis of health data, providing a comprehensive understanding of the user's physiological state. This multi-dimensional approach enables the system to capture complex interactions between different health parameters and their evolution over time, leading to more accurate and personalized health pattern assessments.

In accordance with the embodiment, the artificial intelligence (AI) subsystem 412 further compares the determined health patterns with a predefined baseline to detect a deviation in the real-time health status of the user. The artificial intelligence (AI) subsystem 412 firstly integrates multiple parameters for the predefined baseline. The parameters for these baselines are received from database 306 which integrates multiple data sources. The artificial intelligence (AI) subsystem 412 incorporates expert medical knowledge, including evidence-based guidelines, clinical best practices, expert consensus on normal physiological ranges and indicators of health status, population health data, and user's historical health information for establishing the predefined baseline. These personalized predefined baselines account for the individual's unique physiological characteristics and long-term health trends. This historical data is stored in the database 306.

In accordance with the embodiment, the artificial intelligence (AI) subsystem 412 then compares the determined heath patterns with the predefined baseline to detect a deviation in the user's real-time health status. For instance, the artificial intelligence (AI) subsystem 412 might indicate a deviation as a sudden spike in heart rate or blood pressure that exceeds the user's normal range of variability, indicating potential cardiovascular issues. Gradual trends in salivary biomarkers, such as increasing cortisol levels over time, may indicate the onset of chronic stress or metabolic changes. Alterations in circadian rhythms of temperature or hormone levels, such as persistent elevations in body temperature or disruptions in hormonal cycles, could signal an underlying health issue like infection or inflammation. The artificial intelligence (AI) subsystem 412 can distinguish between normal physiological fluctuations and clinically significant deviations. This capability is crucial for minimizing false alarms while ensuring that subtle, yet important changes are not overlooked.

In another embodiment, upon detection of a deviation, the artificial intelligence (AI) system 412 analyzes the context and potential implications. For instance, a slight elevation in body temperature might be interpreted differently based on the time of day, recent physical activity, or concurrent changes in other parameters. This contextual interpretation allows the artificial intelligence (AI) subsystem 412 to make more informed decisions about whether a medication adjustment is necessary or if further monitoring is warranted. Moreover, the artificial intelligence (AI) subsystem comparative analysis is not static but adaptive. As it accumulates data iteratively about the user's health patterns over time, it can refine and update the personalized baselines, making the system increasingly sensitive to the individual's unique physiological responses and health trajectories. This dynamic, multi-faceted approach to health pattern analysis enables the precision medication control unit to provide highly personalized and responsive medication management. By quickly identifying deviations that may indicate changes in health status or medication needs, the system can initiate timely interventions, potentially preventing the exacerbation of health issues and optimizing therapeutic outcomes.

In accordance with the embodiment, the plurality of subsystems 310 further comprises a dosage prediction subsystem 414. The dosage prediction subsystem 414 is configured to determine a precision dosage instruction upon detection of a deviation in the real-time health status of the user. Upon detecting a deviation in the user's real-time health status, the dosage prediction subsystem 414 initiates the process of determining the appropriate medication dosage. The dosage prediction subsystem 414 begins by receiving a plurality of historical patient data from the database 306. This historical patient data includes at least one of the following: historical health parameters of the user, such as heart rate variability, oxygen saturation levels, body temperature trends, salivary biomarkers (e.g., glucose, cortisol, electrolyte levels), respiratory patterns, and ECG or PPG waveforms; historical medication dosage data, which records the types, amounts, and frequencies of medications previously administered to the user; and treatment responses to previous medications, including observed efficacy, side effects, and any adverse reactions.

The dosage prediction subsystem 414 then employs one or more learning models to process this complex information. Supervised learning algorithms, such as Random Forests and Support Vector Machines, are employed for the prediction. These models are trained on extensive datasets of patient outcomes, allowing them to recognize intricate relationships between patient characteristics, medication dosages, and treatment efficacy. For example, Random Forests, an ensemble learning method, excels at handling high-dimensional data and can capture non-linear relationships between variables, such as how specific biomarker levels (e.g., glucose or cortisol) correlate with medication responses. Support Vector Machines, on the other hand, are particularly effective at finding optimal decision boundaries in complex feature spaces, making them well-suited for classifying patient responses to different dosage regimens, such as determining the optimal dosage for a user based on their heart rate variability or respiratory patterns.

In an exemplary embodiment, the dosage prediction subsystem 414 also incorporates reinforcement learning models. These learning models add a dynamic, adaptive component to the dosage prediction process. By continuously learning from the outcomes of previous dosing decisions, reinforcement learning algorithms can refine and optimize dosage strategies over time. This approach allows the system to adapt to changes in patient physiology, evolving health conditions, and varying environmental factors that may influence medication efficacy.

The training dataset for these models is gathered from multiple sources to ensure comprehensive and reliable performance. The dataset components include anonymized patient records, which provide real-world data on how specific health parameters (e.g., heart rate, temperature, salivary biomarkers) respond to different medication dosages; clinical trial data, which offers controlled and detailed insights into the efficacy and safety of medications across diverse patient populations; and published medical literature, which establishes a foundation of general medical knowledge and dosing principles for specific health parameters and conditions. This broad training base helps establish a foundation of general medical knowledge and dosing principles. Following initial training, the models undergo a fine-tuning phase using more specific datasets relevant to the particular medications and conditions being managed. This phase may involve data from specialized clinical studies or real-world evidence gathered from similar patient populations, focusing on health parameters such as glucose levels, heart rate variability, or respiratory patterns.

The trained models are then tested on held-out datasets not used during training. For instance, the models are tested on datasets that include rare or extreme cases of health parameters, such as unusually high or low glucose levels, to ensure they can handle a wide range of scenarios. The testing phase of the models in the dosage prediction subsystem 414 may include various testing methodologies such as k-fold cross-validation, which can help assess the models' performance across different subsets of the health parameters.

In accordance with the embodiment, once the models are validated, the dosage prediction subsystem 414 generates a preliminary dosage instruction based on the real-time health status of the user and the historical patient data. This preliminary dosage instruction is then optimized to determine a precision dosage instruction for the user. The optimization process considers a plurality of user-specific parameters, including at least one of the following: age, weight, metabolism, and other relevant factors. By taking into account these user-specific parameters, the dosage prediction subsystem 414 can tailor the dosage instruction to the individual needs of the user, ensuring maximum efficacy and minimal adverse effects.

The precision dosage instruction determined by the dosage prediction subsystem 414 includes detailed specifications such as the precise amount of medication to be released, the rate of release of the medication, and the timing of release of the medication. This level of precision ensures that the medication is delivered in a manner that is optimally suited to the user's current health status and physiological needs.

In accordance with the embodiment, the digital controller unit then releases a microfluidic dosage of medication based on the generated optimized precision dosage instruction. The digital controller unit is also configured to adjust the rate of flow and timing of flow of the microfluidic medication based on the precision dosage instruction, ensuring maximum efficacy and minimal adverse effects.

In an alternate embodiment, the system integrates advanced safeguards to prevent accidental overdosing or unauthorized medication release, ensuring patient safety and treatment efficacy. Secure authentication mechanisms, including multi-factor authentication and biometric verification, restrict access to critical functions, while unique cryptographic keys prevent unauthorized control. Dosage control algorithms enforce maximum daily dose limits, implement time-locked dosing schedules to prevent rapid successive doses, and adapt based on patient usage patterns. The system continuously analyzes real-time data to detect anomalies and flag potential risks, preventing unintended medication release.

In accordance with the embodiment, to further enhance medication safety, the system incorporates fail-safe mechanisms, including mechanical dose limiters that regulate the maximum allowable release per cycle and redundant sensors that verify administration accuracy. If connectivity between system components is lost, an automatic shut-off is triggered to prevent uncontrolled dosing. Tamper-evident designs, such as physical seals, logging of unauthorized access attempts, and motion detection to identify unusual device manipulation, further strengthen security. Additionally, advanced encryption ensures the integrity of drug release commands, while blockchain-inspired logging provides auditability and traceability of all medication-related transactions.

In yet another embodiment, patient-specific calibration allows dose customization based on individual factors such as weight, metabolism, and medical history. Regular recalibrations during medical check-ups ensure that dosing remains aligned with the patient's changing health status. The system also integrates drug interaction checking through electronic health records to prevent adverse reactions. Emergency override protocols require a secure, multi-step verification process before any emergency dose can be administered, with immediate alerts sent to healthcare providers for oversight and intervention.

In an exemplary embodiment, the system also incorporates comprehensive patient monitoring and performance metrics to enhance treatment outcomes. Key clinical performance metrics include treatment adherence rate, symptom reduction, and improvements in quality of life. AI-driven analysis of vital signs, such as blood pressure, blood glucose levels, and respiratory function, ensures dynamic adjustments to medication dosing. Patient engagement and satisfaction are also tracked through integrated feedback mechanisms, allowing healthcare professionals to refine treatment strategies based on real-world usage patterns.

The system employs optimized data transmission protocols to minimize latency, edge computing for real-time processing, and intelligent load balancing to prevent network congestion. Data privacy is safeguarded through end-to-end encryption, anonymization techniques, and compliance with regulations such as HIPAA and GDPR. Cybersecurity measures, including intrusion detection, malware protection, and digital signatures, ensure the integrity and confidentiality of patient data, while continuous security audits further strengthen system resilience.

In an alternative embodiment, enhanced safety mechanisms ensure optimal device operation and risk mitigation. The palatal medication delivery device is designed with biocompatible materials to prevent allergic reactions, and dose control algorithms prevent overdosing or underdosing. AI-driven allergy detection analyzes patient history and biomarkers to preemptively flag potential sensitivities. Additionally, failure mode and hazard analysis techniques are employed to proactively identify risks, while continuous patient monitoring and adaptive AI-driven interventions help maintain precise, safe, and effective medication delivery.

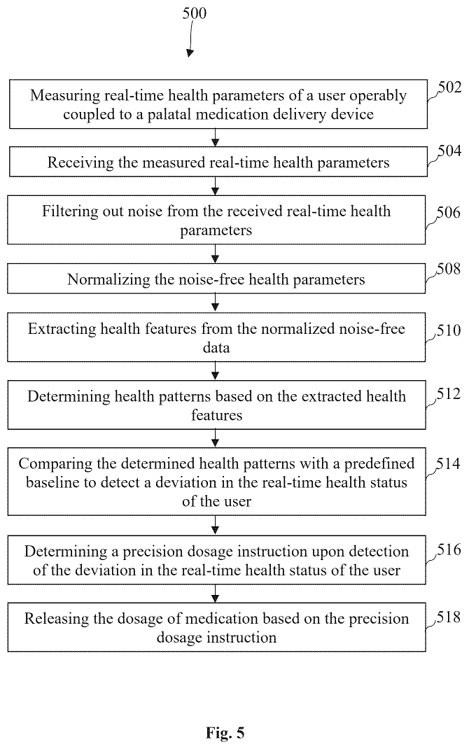

FIG. 5 illustrates an exemplary flowchart 500 of a method for precision oral medication delivery based on real-time health monitoring, in accordance with an embodiment of the present invention.

At step 502, the method measures a real-time health parameter of a user.

At step 504, the method receives the measured real-time health parameters.

At step 506, the method filters out noise from the received real-time health parameters.

At step 508, the method normalizes the noise-free health parameters.

At step 510, the method extracts health features from the normalized noise-free data.

At step 512, the method determines health patterns based on the extracted health features.

At step 514, the method compares the determined health patterns with a predefined baseline to detect a deviation in the real-time health status of the user.

At 516, the method determines a precision dosage instruction upon detection of the deviation in the real-time health status of the user.

At 518, the method releases the dosage of medication based on the precision dosage instruction.

Numerous advantages of the present disclosure may be apparent from the discussion above. The system and method for precision oral medication delivery based on real-time health monitoring may provide significant technical advantages and economic benefits. By leveraging advanced microfluidics, miniaturized sensors, artificial intelligence, and wireless communication technologies, the system enables precise, adaptive medication dosing tailored to individual patient needs. This approach enhances treatment efficacy, reduces side effects, and ensures consistent drug levels in the body, potentially improving patient outcomes and quality of life. The system's ability to continuously monitor real-time health data and dynamically adjust dosages may minimize the need for manual interventions, optimizing resource allocation in healthcare settings. Additionally, the integration of AI-driven analysis ensures accurate and timely dosing decisions, reducing the risk of medication errors and enhancing patient safety.

From an economic perspective, this system may offer a competitive advantage to healthcare providers by enabling personalized, adaptive treatment that improves patient outcomes and reduces healthcare costs. The improved treatment efficacy and reduced side effects may lead to fewer hospitalizations, lower healthcare utilization, and better management of chronic conditions. The system's ability to provide real-time health data and insights may also support strategic decision-making and resource allocation in healthcare settings, potentially leading to more informed investments in medical technologies and practices. Furthermore, the system's wireless communication capabilities facilitate remote monitoring and data sharing, enabling timely interventions and enhancing overall patient care.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A system for precision oral medication delivery based on real-time health monitoring, the system comprising:
a palatal medication delivery device comprising:
a biocompatible layer;
a medication holding chamber comprising a plurality of micro-holes that are distributed across surface of the medication holding chamber; and
a digital controller unit connected with the medication holding chamber configured to control release of medication through the plurality of micro-holes;
a plurality of sensors operably coupled to the palatal medication delivery device configured to measure real-time health parameters of a user,
wherein the real-time health parameters comprise at least one of a body temperature parameter, a heart rate parameter, an oxygen saturation level parameter, and a salivary biomarker parameter;
a precision medication control unit operably coupled to the palatal medication delivery device, comprising:
one or more hardware processors; and
a memory coupled to the one or more hardware processors, wherein the memory comprises a plurality of subsystems in the form of programmable instructions executable by the one or more hardware processors, and wherein the plurality of subsystems comprises:
a data processing subsystem configured to:
receive the measured real-time health parameters;
filter out, by one or more filtering techniques, noise from the received real-time health parameters;
normalize, by one or more normalization techniques, the noise-free health parameters;
extract, by one or more feature extraction techniques, health features from the normalized noise-free data;
an artificial intelligence (AI) subsystem configured to:
determine, by one or more neural networks, health patterns based on the extracted health features,
wherein the determined health patterns comprise at least one of a heart rate variability pattern, a glucose level fluctuation pattern, a physical activity pattern, a respiratory pattern, and a temperature level pattern,
wherein the determined health patterns indicate the real-time health status of the user; and
compare the determined health patterns with a predefined baseline to detect a deviation in the real-time health status of the user;
a dosage prediction subsystem configured to determine a precision dosage instruction, upon detection of the deviation in the real-time health status of the user; and
the digital controller unit configured to release a microfluidic dosage of medication based on the precision dosage instruction.

2. The system as claimed in claim 1, wherein the biocompatible layer is made of at least one of a medical-grade silicone, biocompatible polymers, and hydrogel.

3. The system as claimed in claim 1, wherein the digital controller unit is configured to adjust the rate of medication released through the micro-holes, based on the precision dosage instruction.

4. The system as claimed in claim 1, wherein the plurality of sensors comprises at least one of:
a temperature sensor for measuring body temperature;
a photoplethysmography (PPG) sensor for measuring heart rate and oxygen saturation levels;
an electrochemical sensor for detecting salivary biomarkers including glucose level, cortisol level, and electrolyte level; and
an inertial measurement unit (IMU) sensor for tracking physical activity and movement patterns.

5. The system as claimed in claim 1, wherein the dosage prediction subsystem is configured to determine the precision dosage instruction, upon detection of the deviation in the real-time health status of the user, further comprises:
receiving a plurality of historical patient data from a patient database,
wherein the historical patient data includes at least one of a historical health parameter of the user, a historical medication dosage data, and a treatment response to the medication;
determine, by one or more learning models, a medication dosage instruction based on the real-time health status of the user and the plurality of historical patient data;
optimize the medication dosage instruction to determine a precision dosage instruction for the user, based on a plurality of user-specific parameters,
wherein the user-specific parameters include at least one of an age, a weight, and a metabolism, and
wherein the precision dosage instruction comprises one of a precise amount of medication to be released, a rate of release of the medication, and a timing of release of the medication.

6. The system as claimed in claim 1, further comprises a user interface operably connected to the precision medication control unit configured to display real-time health status and medication dosage information to the user.

7. The system as claimed in claim 1, further comprises a wireless communication unit configured to transmit real-time health data and dosage information to a healthcare provider.

8. The system as claimed in claim 1, wherein the one or more filtering techniques comprise at least one of a Kalman filtering technique and a Butterworth filtering technique.

9. The system as claimed in claim 1, wherein the one or more normalization techniques comprise at least one of a Min-Max Normalization and Z-Score Normalization.

10. The system as claimed in claim 1, wherein the one or more feature extraction techniques comprise at least one of a Fast Fourier Transform (FFT) and a wavelet analysis.

11. The system as claimed in claim 1, wherein the one or more neural networks comprise at least one of a convolutional neural network (CNN) and a recurrent neural network (RNN).

12. The system as claimed in claim 5, wherein the one or more learning models comprise at least one of a supervised learning model and an reinforcement learning model, wherein the supervised learning model includes at least one of a Random Forests model and a Support Vector Machines model.

13. A method for precision oral medication delivery based on real-time health monitoring, the method comprising:
measuring, by a plurality of sensors, real-time health parameters of a user operably coupled to a palatal medication delivery device,
wherein the real-time health parameters comprise at least one of a body temperature parameter, a heart rate parameter, an oxygen saturation level parameter, and a salivary biomarker parameter;
receiving, by a data processing subsystem, the measured real-time health parameters;
filtering out, by one or more filtering techniques, noise from the received real-time health parameters;
normalizing, by one or more normalization techniques, the noise-free health parameters;
extracting, by one or more feature extraction techniques, health features from the normalized noise-free data;
determining, by one or more neural network, health patterns based on the extracted health features,
wherein the determined health patterns comprise at least one of a heart rate variability pattern, a glucose level fluctuation pattern, an activity pattern, a respiratory pattern, and a temperature level pattern, and
wherein the determined health patterns indicate the real-time health status of the user;
comparing, by an artificial intelligence (AI) subsystem, the determined health patterns with a predefined baseline to detect a deviation in the real-time health status of the user;
determining, by a dosage prediction subsystem, a precision dosage instruction upon detection of the deviation in the real-time health status of the user;
releasing, by the digital controller unit, a microfluidic dosage of medication based on the precision dosage instruction.

14. The method as claimed in claim 13, wherein the palatal medication delivery device comprises a biocompatible layer made of at least one of a medical-grade silicone, biocompatible polymers, and hydrogel.

15. The method as claimed in claim 13, further comprising:
adjusting, by the digital controller unit, the rate of medication released through the micro-holes based on the precision dosage instruction.

16. The method as claimed in claim 13, wherein the plurality of sensors comprise at least one of:
a temperature sensor for measuring body temperature;
a photoplethysmography (PPG) sensor for measuring heart rate and oxygen saturation levels;
an electrochemical sensor for detecting salivary biomarkers including glucose level, cortisol level, and electrolyte level; and
an inertial measurement unit (IMU) sensor for tracking physical activity and movement patterns.

17. The method as claimed in claim 13, wherein determining the precision dosage instruction, upon detection of the deviation in the real-time health status of the user, further comprises:
receiving a plurality of historical patient data from a patient database,
wherein the historical patient data includes at least one of a historical health parameter of the user, a historical medication dosage data, and a treatment response to the medication;
determining, by one or more learning model, a medication dosage instruction based on the real-time health status and the historical patient data; and
optimizing the medication dosage instruction to determine a precision dosage instruction for the user, based on a plurality of user-specific parameters,
wherein the user-specific parameters include at least one of an age, a weight, and a metabolism, and wherein the precision dosage instruction comprises at least one of a precise amount of medication to be released, a rate of release of the medication, and a timing of release of the medication.

18. The method as claimed in claim 13, further comprising:
displaying, via a user interface operably connected to a precision medication control unit, real-time health parameters and dosage information to the user.

19. The method as claimed in claim 13, further comprising:
transmitting, via a wireless communication unit, real-time health data and dosage information to a healthcare provider.

20. The method as claimed in claim 13, wherein the one or more filtering techniques comprise at least one of a Kalman filtering technique and a Butterworth filtering technique.

21. The method as claimed in claim 13, wherein the one or more normalization techniques comprise at least one of a Min-Max Normalization and Z-Score Normalization.

22. The method as claimed in claim 13, wherein the one or more feature extraction techniques comprise at least one of a Fast Fourier Transform (FFT) and a wavelet analysis.

23. The method as claimed in claim 13, wherein the one or more neural networks comprise at least one of a convolutional neural network (CNN) and a recurrent neural network (RNN).

24. The method as claimed in claim 17, wherein the one or more learning models comprise at least one of a supervised learning model and a reinforcement learning model, wherein the supervised learning model includes at least one of a Random Forests model and a Support Vector Machines model.
